SEQUENCE LISTING (23 sequences)

SEQ ID NO: 1 - protein, 20 aa, respiratory syncytial virus
KLNNLLEGNDSDNDLSLEDF

SEQ ID NO: 2 - protein, 20 aa, respiratory syncytial virus
KLCGMLLITEDANHKFTGLI

SEQ ID NO: 3 - protein, 20 aa, respiratory syncytial virus
QFPHFSSVVLGNAAGLGIMG

SEQ ID NO: 4 - protein, 18 aa, respiratory syncytial virus
KLCGMLLITEDANHKFTG

SEQ ID NO: 5 - protein, 16 aa, respiratory syncytial virus
KLCGMLLITEDANHKF

SEQ ID NO: 6 - protein, 10 aa, respiratory syncytial virus
KLCGMLLITE

SEQ ID NO: 7 - protein, 20 aa, respiratory syncytial virus
QLLSSSKYTIQRSTGDSIDT

SEQ ID NO: 8 - protein, 20 aa, respiratory syncytial virus
LGIMGEYRGTPRNQDLYDAA

SEQ ID NO: 9 - protein, 20 aa, respiratory syncytial virus
LYDAAKAYAEQLKENGVINY

SEQ ID NO: 10 - protein, 20 aa, respiratory syncytial virus
KLCGMLLITEDANHKFTGLI

SEQ ID NO: 11 - protein, 20 aa, respiratory syncytial virus
GKEMKFEVLTLASLTTEIQI

SEQ ID NO: 12 - protein, 20 aa, respiratory syncytial virus
HDSPDCGMIILCIAALVITK

SEQ ID NO: 13 - protein, 20 aa, respiratory syncytial virus
GIFAGLFMNAYGAGQVMLRW

SEQ ID NO: 14 - protein, 18 aa, respiratory syncytial virus
CGMLLITEDANHKFTGLI

SEQ ID NO: 15 - protein, 16 aa, respiratory syncytial virus
MLLITEDANHKFTGLI

SEQ ID NO: 16 - protein, 14 aa, respiratory syncytial virus
LITEDANHKFTGLI

SEQ ID NO: 17 - protein, 12 aa, respiratory syncytial virus
TEDANHKFTGLI

SEQ ID NO: 18 - protein, 10 aa, respiratory syncytial virus
DANHKFTGLI

SEQ ID NO: 19 - protein, 18 aa, respiratory syncytial virus
KLCGMLLITEDANHKFTG

SEQ ID NO: 20 - protein, 16 aa, respiratory syncytial virus
KLCGMLLITEDANHKF

SEQ ID NO: 21 - protein, 14 aa, respiratory syncytial virus
KLCGMLLITEDANH

SEQ ID NO: 22 - protein, 12 aa, respiratory syncytial virus
KLCGMLLITEDA

SEQ ID NO: 23 - protein, 10 aa, respiratory syncytial virus
KLCGMLLITE

(19) United States
(12) Patent Application Publication
(10) Pub. No.: US 2004/0197338 A1
(43) Pub. Date: Oct. 7, 2004

(54) Respiratory syncytial virus

(76) Inventor: Robert Paul Yeo, Glasgow (GB)

Correspondence Address: Daniel A Monaco, Drinker Biddle & Reath, One Logan Square, 18th & Cherry Streets, Philadelphia, PA, 19103-6996, US

(21) Appl. No.: 10/480,965

(22) PCT Filed: May 14, 2002
(86) PCT No.: PCT/GB02/02265

(30) Foreign Application Priority Data
Jun. 9, 2001 (GB) 0114107.6

(51) Int. Cl. A61K 39/42; C07K 16/10

(52) U.S. Cl. 424/159.1; 530/388.3

(57) ABSTRACT

Respiratory syncytial virus, or RSV, is a cause of respiratory tract infection in humans and other animals. Replication of the virus relies in part upon the association of the nucleocapsid (N) protein with a phosphoprotein (P protein). The present invention describes the identification and sequencing of peptide fragments which bind to the P protein and which disrupt the interaction of the N and P proteins. Such peptides may be used to inhibit replication of RSV, and for treatment of patients infected with RSV. Further aspects of the invention relate to the use of such peptides in the diagnosis of RSV infection.

The present invention relates in part to molecular agents which inhibit the growth and reproduction of Respiratory Syncytial Virus (RSV) and related viruses. The invention relates particularly, but not exclusively, to peptide inhibitors of RSV, and further relates inter alia to agents for treatment of RSV infection, and to agents and kits for diagnosis of the presence of RSV in a test sample.

Respiratory Syncytial Virus (RSV) is a major cause of lower respiratory tract infection in humans, particularly infants, and has been implicated as a cause of respiratory failure in elderly institutionalised people. Infection with RSV manifests in adults as influenza-like symptoms, and typically persists for around 3-5 days. In Western countries, the infection is rarely lethal in otherwise healthy adults, while infection is more likely to result in death in adults in developing countries. Infants of 0-6 months of age are also a high risk group, since antibodies to the virus are unlikely to be present in maternal milk as outbreaks of the virus tend to be cyclical in nature.

The virus is classified in the genus Pneumovirus, family Paramyxoviridae, order Mononegavirales, and is related to a number of other economically-important viruses in addition to human RSV, bovine and ovine RSV species are found, and the related turkey rhinotracheitis virus and avian paramyxovirus affect avian species.

Several attempts have been made in the past to produce an effective treatment for the virus, either to prevent infection, or to reduce the severity or duration of any infection; however, all such treatments so far developed have disadvantages. Vaccination with inactivated RSV has in fact been found to exaggerate the effects of the disease. The development of a widely effective vaccine has been hindered by the rapid mutation rate of the viral surface antigens. The most effective current treatment relies on administering humanised antibodies to the patient; however, even this treatment is not completely effective, and it is relatively expensive.

It is among the objects of embodiments of the present invention to provide an alternative treatment for RSV infection.

The RSV virus has an RNA genome which, in vivo, is coated with multiple copies of the nucleocapsid (N) protein to form a helical structure. During transcription or replication of the RNA genome, the N protein remains associated with the genomic RNA, and is also associated with other viral proteins, among them phosphoprotein (P), M2 (22K), and L protein, which together comprise the transcriptase complex. The genome is only able to undergo transcription or replication when coated with the N protein.

It has also been found that free N protein will non-specifically bind any RNA molecule; when complexed with the P protein, however, the N-P complex specifically binds only genomic RNA. It is believed, therefore, that P protein interacts with free N protein to prevent the illegitimate assembly of nucleocapsid around non-viral RNA. Thus, one of the functions of the P protein may be as a molecular chaperone.

A number of workers have investigated the N-P interaction in other virus systems. It is apparent that, despite the sequence divergence and different sizes of P proteins from the different families and genera of Mononegavirales, the P proteins share common structural features. Sendai virus P protein has two sites of the carboxy terminus and one domain in the amino terminus that have been shown to interact with free N protein, these N protein binding domains having different roles. The amino terminal domain would appear to represent the chaperone function of the P protein, while the carboxy terminal domains serve to bind P protein to the formed nucleocapsids, and may be important for its function as part of the transcriptase complex.

For RSV, two domains in the C-terminal domain of the P protein are involved in the interaction with the N protein, suggesting that the RSV P protein probably has a similar structural layout to other paramyxoviruses. The carboxy 20 amino acids of the RSV P protein have been implicated in binding to N protein, as has a region adjacent to this domain, and possibly the first 40 amino acids of the amino terminus. By analogy to Sendai virus, the amino terminus of the RSV P protein would be responsible for its chaperone function. The carboxy domains may be involved in the binding of the P protein to assembled nucleocapsids as part of the P protein's role in the polymerase complex.

However, previous attempts to carry out similar investigations on the RSV N protein have yielded little information. The N protein of RSV has not lent itself to mutagenesis, suggesting that the N protein has a conformation that is susceptible to misfolding, resulting in an inactive protein after such manipulation.

The present invention relies on the discovery by the applicant of a number of peptide sequences derived from the amino acid sequence of the RSV N protein which possess an ability to interact with the P protein, and the further unexpected discovery that such interactions block the normal interaction between the P protein and the N protein.

According to a first aspect of the present invention, there is provided a compound which binds to respiratory syncytial virus phosphoprotein, and which inhibits binding of said phosphoprotein to respiratory syncytial virus nucleocapsid protein.

Compounds of the present invention therefore have the potential to be used as antiviral preparations against RSV: administration of a compound of the present invention to a patient in a suitable form and quantity would be expected to prevent the formation of the N-P complex, and therefore may be expected to prevent formation of the transcriptase complex of the virus. The virus will therefore be unable to replicate.

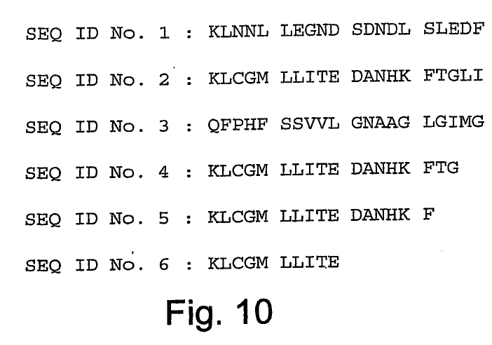

Compounds of the present invention may interact with one or more regions of the RSV phosphoprotein. Conveniently, compounds of the present invention are capable of binding to the carboxy terminus of RSV phosphoprotein. More preferably, such compounds bind to the C-terminal 20 amino acids of the phosphoprotein (given as SEQ ID No. 1 in FIG. 10). This sequence is believed to be one of those involved in N-P interaction; however, additional sequences of the P protein may be of importance, and compounds of the present invention may of course be capable of binding to alternative sequences in addition to or instead of the C-terminal 20 amino acid sequence. Compounds which bind to a peptide comprising said C-terminal 20 amino acids, to a peptide having at least 75%, preferably 90%, and more preferably 95% sequence homology thereto, or to similar amino acid sequences are also included within the scope of the present invention. For example, for the purpose of the present invention “similar” amino acid sequences may include sequences having conservative replacements made between amino acids within the following groups:

(i) Alanine, serine and threonine;

(ii) Glutamic acid and aspartic acid;

(iii) Arginine and lysine;

(iv) Asparagine and glutamine;

(v) Isoleucine, leucine and valine;

(vi) Phenylalanine, tyrosine and tryptophan.

Preferably, a compound according to the present invention comprises a peptide. Such a peptide may comprise the 20 amino acid sequence given as SEQ ID No. 2 (shown in FIG. 10, peptide N4), or sequences with conservative amino acid substitutions, as described above. A peptide of the present invention may alternatively have 80% homology to SEQ ID No.2; or more preferably 90% homology; most preferably 95% homology. Alternatively, peptides according to the present invention may comprise the amino acid sequence given as SEQ ID No. 3 in FIG. 10 (peptide N22). A peptide of the present invention may yet further comprise at least one of the amino acid sequences given as SEQ ID No. 4, 5, or 6 given in FIG. 10 (peptides N4.6, N4.7, and N4.10), or sequences with conservative amino acid substitutions, as described above.

Furthermore, functionally active derivatives derived from the peptide are included within the scope of the present invention. The polypeptide required can be modified in vivo and/or in vitro, for example, by glycosylation, amidation, carboxylation, phosphorylation and/or post-translation cleavage; thus inter alia peptides, oligopeptides, proteins and functionally active derivatives thereof are encompassed thereby.

Alternatively, a compound of the present invention may comprise an antibody, more preferably a monoclonal antibody.

According to an aspect of the present invention, there is provided the use of a compound which binds to respiratory syncytial virus phosphoprotein, and which inhibits binding of said phosphoprotein to respiratory syncytial virus nucleocapsid protein in the preparation of a medicament for the treatment of respiratory syncytial virus infection.

According to a further aspect of the present invention, there is provided a pharmacologically active amount of a compound which binds to respiratory syncytial virus phosphoprotein, and which inhibits binding of said phosphoprotein to respiratory syncytial virus nucleocapsid protein, in combination with a physiologically acceptable carrier.

One embodiment of this aspect of the present invention provides an active compound enclosed in a lipid membrane. Such an embodiment is able to fuse with an animal cell membrane, and deliver the active compound to the cell.

A yet further aspect of the present invention provides a drug delivery device including a pharmacologically active amount of a compound which binds to respiratory syncytial virus phosphoprotein, and which inhibits binding of said phosphoprotein to respiratory syncytial virus nucleocapsid protein, enclosed in a lipid membrane, in combination with a physiologically acceptable carrier therefor.

Preferably the delivery device is designed to administer a pharmacologically active amount of the compound to a patient's respiratory system; preferably to the lungs. For example, the delivery device may comprise a metered inhaler device containing an aerosol formulation of said compound.

The present invention still further provides a kit for the detection of respiratory syncytial virus in a test sample, the kit comprising a compound which binds to respiratory syncytial virus phosphoprotein, and which inhibits binding of said phosphoprotein to respiratory syncytial virus nucleocapsid protein, in combination with any necessary detection and washing reagents.

Further provided by the present invention is a compound which binds to respiratory syncytial virus phosphoprotein, and which inhibits binding of said phosphoprotein to respiratory syncytial virus nucleocapsid protein, said compound being immobilised on a solid support substrate. Such a substrate/compound complex may further be used in a kit in accordance with the above aspect of the invention.

The present invention yet further provides a recombinant cell expressing a peptide which binds to respiratory syncytial virus phosphoprotein, and which inhibits binding of said phosphoprotein to respiratory syncytial virus nucleocapsid protein. Such a cell may be prokaryotic or eukaryotic; preferred embodiments of the invention provide bacterial or mammalian cells expressing such a peptide.

According to a still further aspect of the present invention there is provided the use of a peptide comprising the amino acid sequence given as SEQ ID No.1 in the identification of a compound which binds to respiratory syncytial virus phosphoprotein and which inhibits binding of said phosphoprotein to respiratory syncytial virus nucleocapsid protein. Also provided in accordance with the present invention is a compound identified in such a way. For example, such compounds may be identified by a competition assay, by an antibody assay, by immobilising a peptide on a substrate, or such other methods as will be apparent to those of skill in the art.

Yet further provided in accordance with the present invention is the use of a peptide comprising an amino acid sequence having at least 75%, preferably 90%, more preferably 95% sequence homology to or similar to that given as SEQ ID No. 1 in the identification of a compound which binds to phosphoprotein of a virus related to respiratory syncytial virus.

These and other aspects of the present invention will now be described by way of illustration only, with reference to the following examples and with reference to the accompanying drawings, which show:

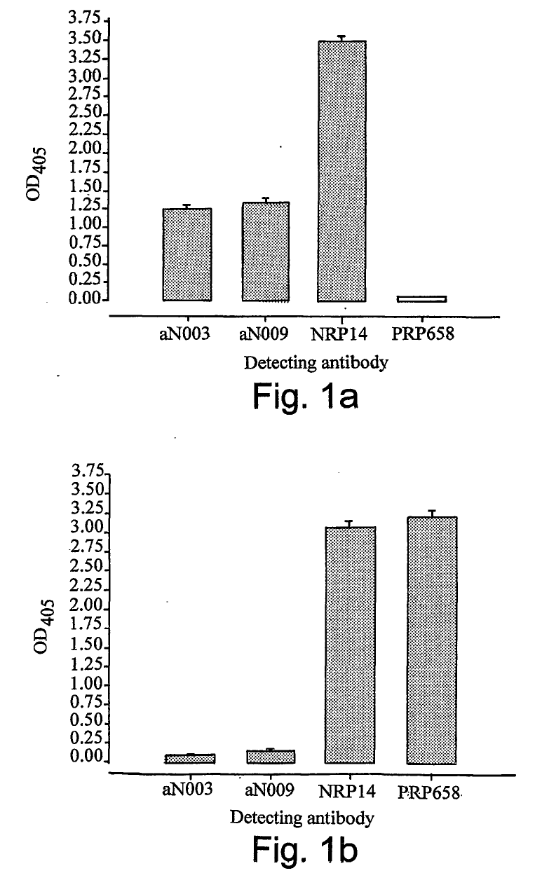

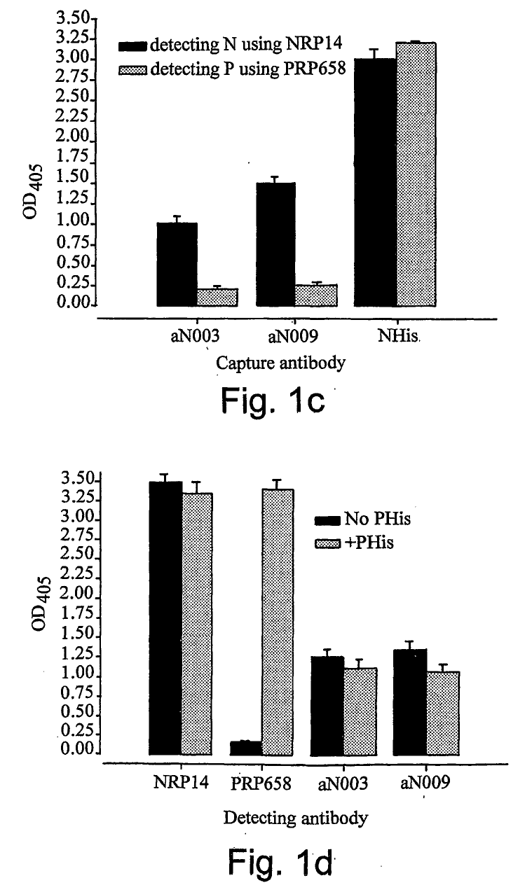

FIG. 1. Effect of Mabs αN003 and αN009 on in vitro binding of N^{His} and P^{His} proteins.

FIG. 1a. αN003 and αN009 were directed against N^{His} immobilized on a microtitre plate. Bound antibody was detected with the appropriate secondary antibody conjugated to HRP.

FIG. 1b. Capturing N^{His} using immobilized P^{His}. P^{His} was bound to the microtitre plate overnight in PBS. After blocking, N^{His} was bound and detected with the polyclonal or monoclonal antibodies. PRP658 detects the P^{His} bound to the plate.

FIG. 1c. Capturing N^{His} using the monoclonal antibodies and subsequent binding of P^{His}. The monoclonals were bound to the microtitre plate overnight as previously described. N^{His} was added and allowed to bind. P^{His} was subsequently added and allowed to bind; bound P^{His} was detected using PRP658 and an anti-rabbit HRP conjugate.

FIG. 1d. Binding of P^{His} and the monoclonal antibodies to immobilized N^{His}. NHis was immobilized onto microtitre plates. PHis was added in saturating quantities and allowed to bind for 1 hour. After washing off unbound PHis, the monoclonals were added and the binding of which was detected using an anti-mouse-HRP conjugate. Binding of PHis was detected using PRP658 and an anti-rabbit-HRP conjugate.

NB: All experiments used saturating quantities of protein which were determined empirically by ELISA experiments (not shown).

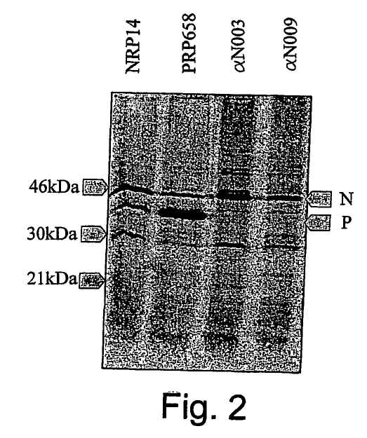

FIG. 2. RIPA of infected cell lysates.

[³⁵S]-Methionine labelled RSV infected cell lysates were subjected to immunoprecipitation using antisera NRP14 or PRP658, or monoclonals αN003 or αN009. The precipitated proteins were subjected to 12% SDS-PAGE. The radiolabelled proteins were detected using a Bio-Rad personal FX phosphorimager. The positions of the molecular weight markers are shown.

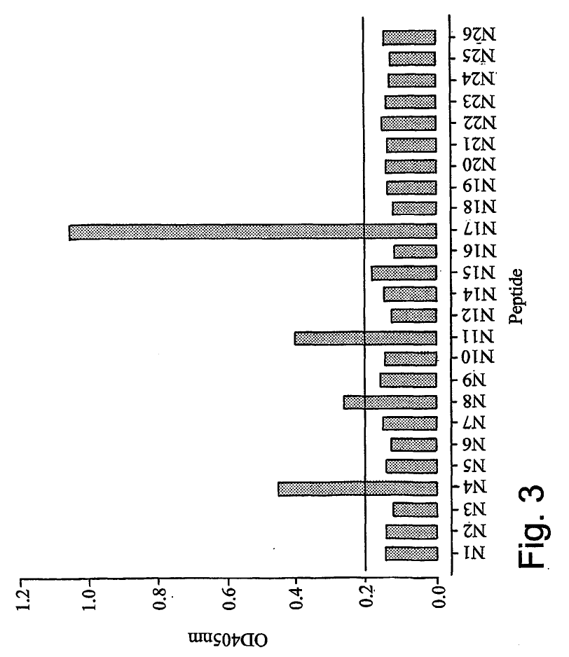

FIG. 3. Reactivity of recombinant P^{His} protein against N specific peptides.

Microtitre plate wells, in quadruplet, were coated with the RSV N peptides overnight in PBS. After blocking, the P^{His} was added at 1 ug/ml and allowed to bind for one hour at room temperature. Captured P^{His} was detected using anti-His tag antibody (Sigma) conjugated to HRP. The background (binding detected in the absence of P^{His}) was determined for each peptide, binding below an OD405 of 0.2 was due to non-specific binding between the peptides and the anti-His antibody.

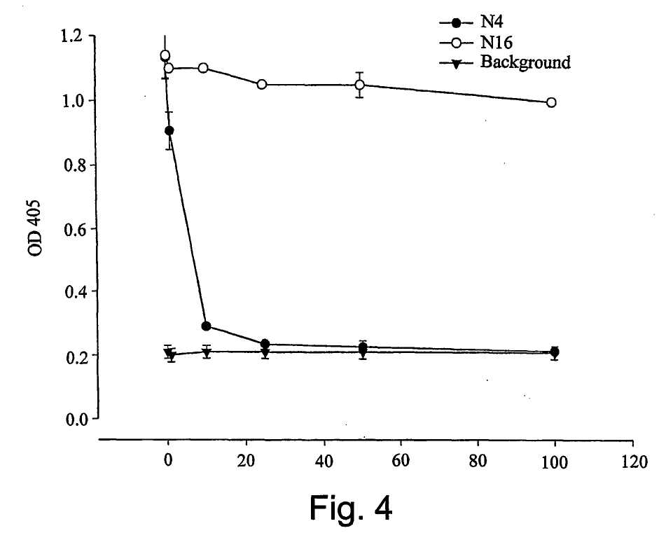

FIG. 4. Blocking of N-P binding by peptide N4.

Microtitre plates were coat with a saturating amount of P^{His} and incubated overnight in PBS. Various amounts (0-100 μg/ml) of peptide N4 or N16 were added for 1 hour. A saturating amount of N^{His} was added, still in the presence of peptide, and allowed to bind. Bound N^{His} was detected using the NRP14 polyclonal antisera and an anti-rabbit-HRP conjugate as described. “Background” represents the reading taken when N^{His} was omitted from the blocking assay.

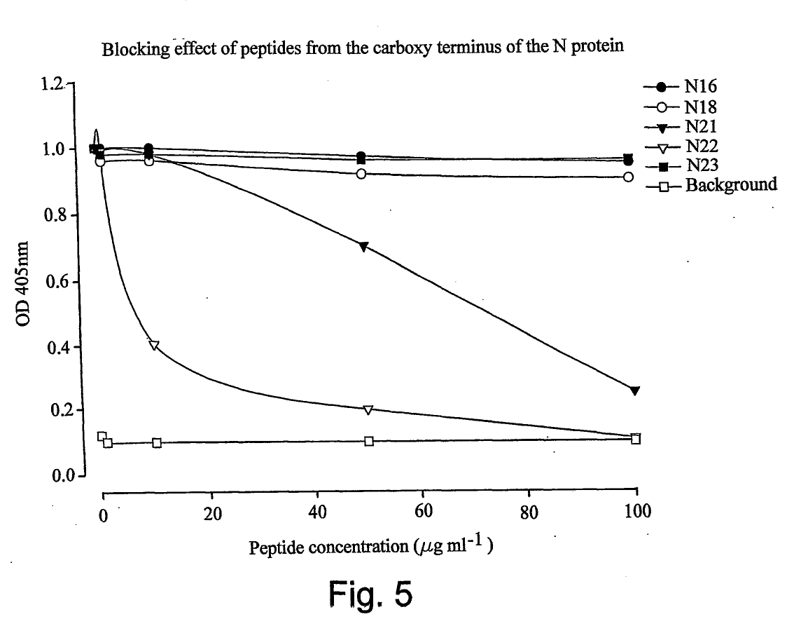

FIG. 5. Blocking of N-P binding by peptide N22.

The experiment giving rise to the results shown in FIG. 4 was repeated with peptide N22.

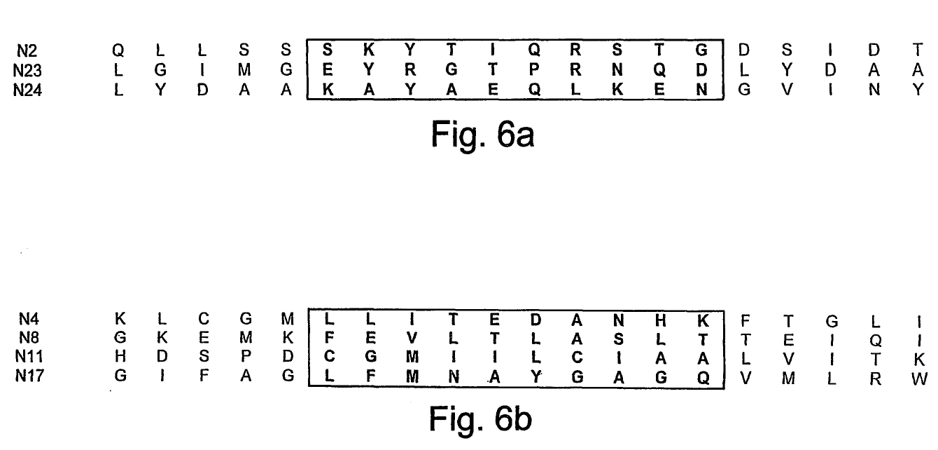

FIG. 6. Sequences of peptides that bound either P^{His} or the monoclonal antibodies in this study.

The amino acids that are boxed in bold print represent the 10 unique amino acids for each peptide. The flanking sequences are shared with the adjacent peptides.

(a) Peptides that reacted against the monoclonals generated in this study.

(b) Peptides that bound P^{His} protein.

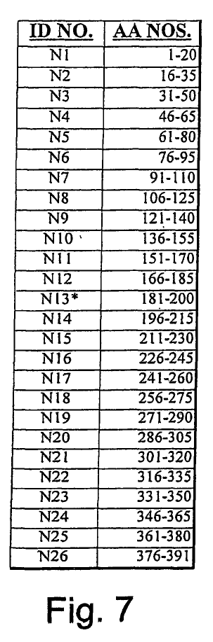

FIG. 7. Listing of the peptides used in this study.

Peptides represent the linear amino acid sequence of the RSV A2 N protein sequence with N1 being the amino terminus and N26 the carboxy terminus. The peptides are 20-mers with a 5 amino acid overlap with the adjacent peptides.

NB *=N13 was not successfully synthesized, and was not included in any assay. It is listed here for continuity therefore 10 amino acids, 186-195 inclusive (2.6% of N protein's length), are missing.

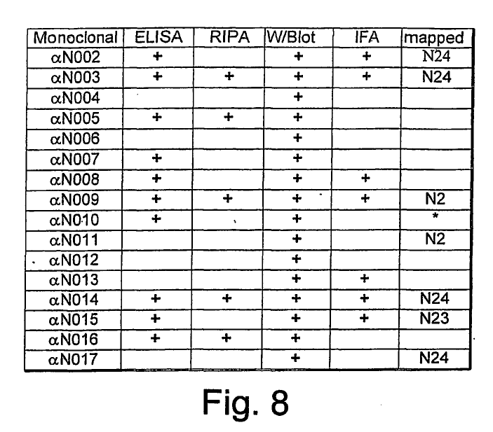

FIG. 8. Listing of the monoclonal antibodies produced, and reactivities in various immunological assays.

W/Blot=western blot; IFA=immunofluorescence.

“Mapped” refers to the peptide that some of the monoclonals reacted to in ELISA. No cross reactivity was observed in the flanking peptides.

*=Monoclonal antibody αN010 could not be accurately mapped to a peptide but deletion analysis of N protein (data not shown) indicates that it binds to an epitope in the first fifty amino acids of the amino terminus of the N protein.

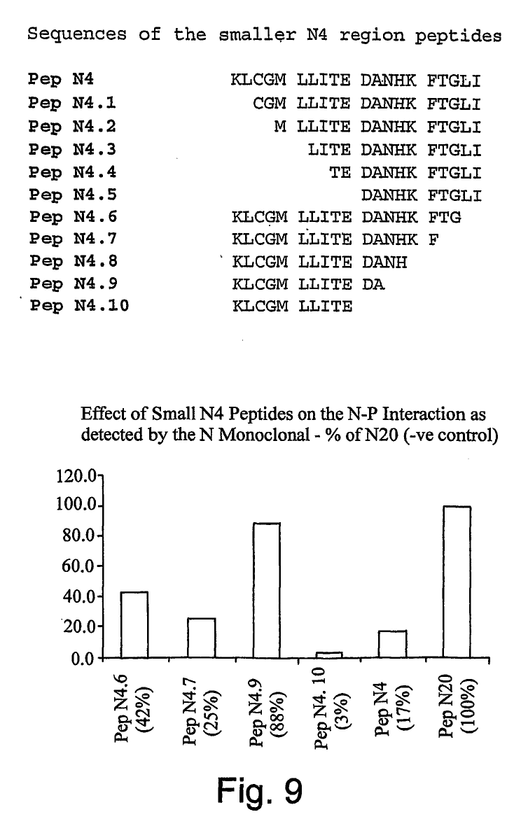

FIG. 9. Effect of small peptides derived from peptide N4 on N-P interaction.

FIG. 10. Selected Peptide Sequences.

This figure shows the C-terminal twenty amino acid sequence of the RSV P protein (SEQ ID No. 1); the twenty amino acid sequence of the N4 peptide (SEQ IQ No. 2); the amino acid sequence of the N22 peptide (SEQ ID No. 3); and a number of shorter amino acid sequences derived from peptide N4 having binding activity for RSV P protein (SEQ ID No.4, 5, and 6).

### MATERIALS AND METHODS

Cells and viruses. CV-1 cells were maintained in Dulbecco's modified Eagles medium (DMEM) supplemented with 10% fetal calf serum. The A2 strain of RSV was used throughout this study. CV-1 cells at 70% confluency were infected at a multiplicity of 0.1, the virus was allowed to bind for 1 hour before it was removed and DMEM with 2% fetal calf serum was added. The infection was allowed to proceed for 36-48 hours at 33° C. until cpe was evident before harvesting for RNA, protein labelling or to produce virus stocks.

Nucleic acid manipulations. Standard methods were followed as described by Sambrook et al. RNA from infected cells was isolated using the Ambion “Totally RNA” kit and stored in water at −70° C. 1 μg of total RNA was reverse transcribed using random hexameric primer with the Boehringer Mannheim AMV RT kit in a 25 μl volume. 1 μl of this was used in RT-PCR with specific primers for the N and P genes of RSV containing appropriate restriction sites for cloning into pET16b (Novagen). Plasmids pETN and pETP were isolated. The inserts were sequenced on an ABI Prism 377 automated sequencer using the Big Dye Terminator cycle sequencing kit (Perkin Elmer Applied Biosystems). The N protein construct had no changes from the sequence available on the Genbank database (accession code M74568 and references contained therein). The P protein construct had one change from the published A2 sequence (amino acid 171) resulting in an Isoleucine to Valine change. This change returns the Genbank A2 M74568 sequence to the consensus sequence at this point when compared to bovine, caprine and ovine strains of RSV (data not shown). The constructs, which would result in the expression of N and P proteins with a Histidine tag at their amino terminus were then used to transform E. coli BL21 (DE3) pLysS.

Purification of recombinant N^{His} and P^{His} proteins. BL21 (DE3) pLysS bacteria with either pETN (expressing N^{His}) or pETP (expressing P^{His}) were grown overnight in 10 ml of 2×YT containing 100 μg/ml ampicillin and 34 μg/ml chloroamphenicol. 1 ml was used to inoculate 100 ml of 2×YT also containing ampicillin and chloroamphenicol. The cultures were allowed to grow to an OD600 of 0.6-0.8 before induction with 1 mM IPTG and expression was allowed to proceed for 4 hours before harvesting. The His-tagged proteins were purified using metal chelate chromatography using Hi-Trap columns and an AKTA purifier10 (Pharmacia) according to manufacturer's conditions. Fractions containing the proteins were pooled, dialyzed against PBS, and concentrated using Centricon 10 concentrators (Millipore) to 1 mg/ml. Protein samples were stored in 15% v/v glycerol at 4° C.

Generation of monoclonal and polyclonal antisera. Polyclonal antisera were raised against N^{His} and P^{His} in New Zealand White rabbits. The polyclonal antiserum NRP14 is reactive against the RSV N protein and shows no reactivity to the His-tag. PRP658 is a polyclonal antiserum against the RSV P protein; cross reactivity to the His-tag was removed by absorbing against acetone powders of BL21 (DE3) pLysS expressing N^{His}. Monoclonal antibodies to the N protein were raised in Balb/c mice. The antibodies used in this study were purified on a Protein G Hi-Trap column (Pharmacia) from hybridoma supernatants.

Production of N specific peptides. Peptides were prepared by standard procedures on a Shimadzu automated peptide synthesizer. Twenty-five overlapping 20-mers representing 97% of the N protein's amino acid sequence (RSV A2 N protein is 391 amino acids long; FIG. 7) were prepared using FMOC chemistry and amino acid substrates from Novabiochem (Scotland). Peptides were analysed for purity and size on a Pharmacia HPLC system. Peptides were stored lyophilised at −20° C. and resuspended in water at 1 mg/ml when needed except for N5, N8, N11, N15 and N17 which required 10% (v/v) acetic acid for solubility.

ELISA studies. In vitro binding of N^{His} and P^{His} proteins were carried out using an ELISA based assay. Microtitre plates (Immulon 2, Dynex Technologies) were coated with saturating amounts of the appropriate protein or purified monoclonal antibodies overnight at 4° C. Plates were washed and free protein binding sites were blocked with 5% (w/v) dried milk powder (Marvel). Proteins and antibodies were added in different orders (see figure legends for details) and detected with the appropriate polyclonal or monoclonal antisera, the binding of which was detected using either anti-mouse or anti-rabbit antibody conjugated to HRP (Sigma). ABTS (1 mg/ml, Sigma) colour reagent in 50 mM Citrate buffer pH4.0 with hydrogen peroxide was added to each well and colour allowed to develop for up to 30 minutes. Washing and binding were all carried out in PBS+0.1% (v/v) Tween-20. The plates were analysed on a Dynex microtitre plate reader at a wavelength of 405 nm (OD405).

Peptide Scanning. To partially map the monoclonal antibody epitopes and the P protein binding domain a peptide scanning protocol was used. Peptides were used to coat the wells of microtitre plates in sextuplet overnight at a concentration of 1 μg/ml in PBS at 4° C. After washing and blocking, either the antibodies or P^{His} protein was added at saturating amounts and allowed to bind for 1 hour at room temperature. Bound ligand was detected using either an anti-mouse antibody conjugated to HRP for bound monoclonal, or an anti-Histidine Tag antibody conjugated to HRP (Sigma) to detect bound P^{His}. Colour development and detection was as described above.

Peptide blocking of N-P binding. P^{His} protein was used to coat the wells of a microtitre plate overnight at 4° C. After washing, the peptides were added at varying concentrations from 10 μg/ml to 100 μg/ml. Each concentration was performed in sextuplet. After binding for 1 hour the plates were washed and N^{His} protein added at saturating quantities and allowed to interact with P^{His}. Bound protein was detected using the anti-N protein polyclonal NRP14 followed by an anti-rabbit HRP conjugate. Colour development was described above.

Radio-immunoprecipitation assay (RIPA). RSV infected CV-1 cells were starved of methionine 36 hours post-infection for one hour before the addition of [35S]-methionine (NEN) at 100 μCi/ml. Cultures were harvested 3 hours later into lysis buffer (50 mM Tris HCl pH8.0, 120 mM NaCl, 0.5% v/v NP40) and clarified by centrifugation in a Beckman benchtop Ultracentrifuge at 50,000×g. 50 μl was used in each immunoprecipitation, 1 μl of monoclonal or polyclonal antisera was added and allowed to complex. The antibody-antigen complexes were precipitated using Pansorbin cells (Calbiochem), washed five times in lysis buffer and analysed by SDS-PAGE. All binding and washing steps were carried out at 4° C. Gels were fixed and dried and the radiolabelled products detected using a Bio-Rad Personal FX phosphorimager.

Results

Production of monoclonal antibodies to the N protein of RSV. For this study we generated a panel of monoclonals to the N protein of RSV. GST-N was purified from BL21 (DE3) pLysS, gel purified using a S&S Biotrap and used as antigen for immunizing Balb/c mice (details not shown). Sixteen hybridomas were obtained that reacted against N from RSV infected CV-1 cells. They were screened in a number of immunological assays, using antigen prepared from RSV infected CV-1 cells, for their ability to detect N protein (see FIG. 8). All the monoclonal antibodies reacted in western blot assays suggesting that they are against linear, rather than conformational, epitopes.

Peptide mapping of the monoclonal antibody epitopes on the N protein. Seven of the ten monoclonals that were reactive in ELISA were subsequently mapped against a panel of 20-mer peptides representing 97% of the N protein (FIG. 7). Amino acids 186-195 were not included, as attempts to make this peptide were unsuccessful. It is evident that there are two immuno-dominant regions on the N protein. Group I represents amino acids 21-30 inclusive (peptide N2) and Group II amino acids 331-365. Group II comprises at least two immunological sites, represented by sequences contained within peptides N23 and N24. The sequences of peptides N2, N23 and N24 are presented in FIG. 6. Further mapping will be necessary to fine map the actual monoclonal antibody epitopes with these peptides.

N-P binding in the presence of the monoclonal antibodies. The use of the monoclonal antibodies as tools in analysing the N-P binding was investigated. One monoclonal antibody was selected from each of the two groups for further analysis. Monoclonal antibodies αN003 and αN009 were purified from hybridoma supernatants by FPLC on a Hi-Trap Protein G column and used to investigate domains on N protein for P protein binding in vitro using ELISA based assays.

(1) Detection of N^{His}. Elisa plates were coated with saturating amounts of N^{His} and bound protein detected with the monoclonal antibodies (αN003 and αN009) or with the anti-N protein polyclonal (NRP14). From FIG. 1a it is evident that the monoclonals and polyclonal show good reactivity to N^{His}. Cross-reactivity with other His-tagged proteins was also investigated. No detectable cross reactivity was apparent with P^{His} or with other His-tagged proteins (data not shown).

(2) P^{His} binding to N^{His} prevents access by the monoclonal antibodies to the N^{His} protein. To test that our purified proteins retained the ability to bind in vitro, ELISA plates were coated with a saturating amount of P^{His} protein overnight. After blocking free protein binding sites on the microtitre plates with Marvel, N^{His} was added at saturating concentrations. Binding of N^{His} to the immobilized P^{His} was detected by the polyclonal antiserum NRP14. However, FIG. 1b shows that there is a significant decrease in reactivity with the monoclonal antibodies when compared to the NRP14 polyclonal suggesting that the binding of N protein to P protein inhibits the monoclonal antibody interactions. Thus it appears that these monoclonals might in fact represent regions of N protein that are involved in the binding to P protein or are in close proximity to the P binding site(s).

(3) The monoclonal antibodies block the N-P interaction in vitro. To determine whether the monoclonal antibodies would block the binding of P^{His} to N^{His} the assay was effectively reversed. Monoclonals αN003 and αN009 were immobilized onto microtitre plates and used to capture N^{His}. P^{His} protein was then allowed to bind to the captured N^{His} and was detected using the anti-P protein polyclonal, PRP658. FIG. 1c illustrates that the binding of PR is appears to be severely reduced by the presence of the monoclonal antibodies confirming the possibility that P protein is binding close to where αN003 and αN009 bind or that the P protein shares the same binding site(s) as the monoclonal antibodies.

(4) The monoclonal antibodies and P^{His} do not share the same binding site. Saturating amounts of N^{His} protein were used to coat an ELISA microtitre plate. P^{His} was added and allowed to bind. After washing off unbound P^{His}, the monoclonal antibodies were added. Binding of the monoclonal antibodies could be shown in the presence of bound P^{His} (the binding of which could be detected by PRP658) within the same sample set. Variations of this experiment were to reverse the order of addition (i.e. monoclonal antibodies first, followed by P^{His}) or to add two ligand simultaneously. Independent of which order the N binding species were added, the binding of one was apparently unaffected by the presence of the other. For example, FIG. 1d demonstrates that the reactivity of monoclonal antibody αN003 or αN009 is not reduced in the presence of P^{His}. The apparent contradictory blocking of the N-P or N-antibody interactions in the previous ELISA experiments can be explained in either one of two ways. Firstly, N^{His} molecules may be immobilized on the plate in a number of orientations exposing or hiding different regions of the N^{His} protein. This might allow binding exclusively to either the monoclonal antibody or P^{His} target sites on different molecules of N^{His}. In contrast, in the previous assays when blocking was observed, the P^{His} or anti-N monoclonal antibodies were used to capture N^{His} from solution. In this instance it is unlikely that any target sites would be hidden and both P^{His} or a monoclonal antibody could potentially interact with all N^{His} molecules. The observed competition in these experiments therefore suggest that the P^{His} and monoclonal antibody binding sites are sufficiently close that satiric hindrance may occur but (taken together with FIG. 1d) are nevertheless not identical.

Radio-immunoprecipitation using monoclonal antibodies αN003 and αN009. Monoclonal antibodies αN003 and αN009 were used in a RIPA assay to determine if they co-immunoprecipitated N and P from virus infected cells. CV-1 cells were infected with the A2 strain of RSV as described earlier. The infected cells were radiolabelled with [³⁵S]-methionine and lysates prepared in RIPA buffer. N and P were precipitated by the monoclonal antibodies to N protein or with the polyclonal antisera to the N or P proteins and subsequently analysed by SDS-PAGE. Gels were dried and visualized using a Bio-Rad personal FX phosphorimager. From FIG. 2 both monoclonal antibodies αN003 and αN009 precipitate N protein, but unlike the polyclonals (NRP14 and PRP658) they do not co-precipitate P protein from the infected lysates. The RIPA confirms the in vitro binding data that αN003 and αN009 only recognize RSV N protein that is not complexed with P protein. An observation made during these experiments was that the salt concentration of the RIPA buffer could effect the co-precipitation of P protein with N protein; in experiments with a higher salt buffer (150 mM compared to 120 mM salt) the N-P complexes were easily disrupted. Under the higher salt conditions the polyclonals also failed to demonstrate co-precipitation of the N-P complex (data not shown). Whether this salt dependent interaction gives an indication of the nature of N-P binding remains to be seen. A number of buffer conditions were employed and at no time did αN003 or αN009 co-precipitate P protein with N protein from infected cell lysates (data not shown).

Peptide mapping of the P binding site on the N protein. The N peptides previously used to map the monoclonal antibodies were used to identify amino acid sequences on N protein that contribute to the binding of P protein. The peptides were used to coat microtitre plates as previously described. P^{His} protein was added at saturating quantities and allowed to bind for one hour. The plates were washed and bound P^{His} detected using anti-His antibody which detects the amino terminus histidine tag. From a number of experiments it was determined that peptides with binding values of less than an OD405 of 0.2 represented background binding caused mainly by cross reactivity of the anti-His tag antibody and the peptides. From FIG. 3 it is evident that P^{His} binds to peptides N4, N8, N11 and N17 above an OD405 of 0.2. The sequences of the P^{His} binding peptides are given in FIG. 6. The distribution of the peptides enforces the concept that the P protein binding site on N protein involves a number of distinct regions on the N protein that fold together to form a single P protein binding domain or alternatively they may be separate regions that independently bind P protein.

P^{His} binding was variable between the different peptides (compare N4 to N17) suggesting that the various regions of the N protein contribute different proportions to the affinity constant of the N-P interaction. The RSV P protein has a number of N protein binding sites that would appear to be used for different functions depending on whether N protein is complexed on the nucleocapsid or in the soluble form (N⁰). This may account for the number of P protein binding sites on the N protein.

N peptide inhibition of the RSV N-P interaction. It was of interest to determine if any of the peptides that bound P^{His} protein could inhibit binding of N^{His} protein to P^{His} protein. Initial attempts with peptides N8, N11 and N17 proved problematic due to solubility problems in water, requiring that an unsuitable buffer (containing 0.01% v/v acetic acid in the 1 μg/ml concentration) was used. These buffer conditions prevented N-P binding even in the absence of peptide. Thus only N4 was suitable, as it was soluble in PBS and bound P^{His} (FIG. 3). As a negative control peptide N16, which does not bind P^{His}, was used (see FIG. 3)

P^{His} was used to coat microtitre plates. After washing and blocking free protein binding sites the plates were incubated with differing amounts of N4 or N16 peptide (0 μg/ml<100 μg/ml) for 1 hour at room temperature. A saturating amount of N^{His} was added to the wells for 1 hour at room temperature. Bound N^{His} protein was detected using the anti-N protein rabbit polyclonal, αNRP14, and an anti-rabbit HRP conjugate. FIG. 4 shows that the presence of peptide N4 caused a significant decrease in the binding of N^{His} to immobilized P^{His} protein. The binding was reduced to background levels at a relatively low peptide concentration with the major drop in binding occurring at 10 μg/ml (approximately 4.5×10⁻⁶ M). N16 has no effect on the in vitro binding of N to P protein. Thus, in this assay peptide N4 would appear to have a blocking activity on N-P binding. Peptide N4 may be able to bind to P^{His} preventing access by N^{His} to P^{His}'s binding site. The N4 peptide may include a sequence crucial for the RSV N-P interaction.

Similar experiments were repeated with peptides N16, N18, N21, N22, and N23, the results of which are shown in FIG. 5. These results demonstrate that peptide N22 is also capable of disrupting N-P interaction.

Discussion.

The interaction between the N and P proteins of RSV has been investigated in vitro using monoclonal antibodies to the N protein and a set of peptides representing 97% of the N protein amino acid sequence (FIG. 7).

A number of monoclonal antibodies have been raised against a GST-N fusion protein (details not shown) to aid in the structural analysis of the RSV N protein. They were tested in a number of immunological assays for reactivity against N protein produced in RSV infected CV-1 cells (FIG. 8). All were reactive in western blot assay indicating that they are most likely targeted against linear epitopes. Of the sixteen available seven could be mapped to one of two domains (represented by peptides N2: a/a 16-35, N23: a/a 331-350 and N24: a/a 346-365, FIGS. 6 and 8). The monoclonal antibodies do not react with the adjacent peptides suggesting that the actual epitopes lie within the ten unique amino acids of each of the peptides. Most of the monoclonal antibodies that were mapped (5/7) by peptide scanning were located in the carboxy domain. The anti-nucleocapsid protein polyclonal antiserum, αNRP14, also binds preferentially to the carboxy terminus of the RSV N protein (data not shown), indicating that the carboxy domain of the RSV N protein contains dominant B cell epitopes. The amino and carboxy termini of the RSV N protein have previously been shown to be predominant immunological targets in the sera of convalescent patients. More work is under way to characterize the monoclonal antibodies that have been partially mapped. The monoclonal antibodies should help in structural studies of the N protein in nucleocapsid assembly.

Further mapping has been performed using shorter peptides to locate the actual epitope within the peptide N4, the results of which are given in FIG. 9. This shows that three of the shorter peptides, N4.6, N4.7, and N4.10 have a significant effect on N-P interaction. N4.9 is shown as an example of a shorter peptide without a significant effect, while N20 was included as a negative control. It is observed that common to all active peptides is a ten amino acid sequence, KLCGM LLITE.

Using two of the monoclonal antibodies generated in this study, one from the amino domain (αN009) and the other from the carboxy region (αN003) we investigated the binding of RSV N protein to the P protein. Previous reports had implicated a role for each of these domains in the interaction of N protein with the P protein. We observed blocking of the N-P interaction with these monoclonal antibodies (FIGS. 1b and 1c) which agrees with previous findings. Krishnamurthy and Samal (J. Gen. Virol. 79:1399-1403, 1998) investigated the bovine RSV N-P interaction using the yeast two hybrid system. Small deletions (up to 31 amino acids) at the carboxy terminus resulted in loss of P protein binding, as did an independent 40 amino acid deletion of the amino terminus. However, binding was regained if both the amino and carboxy termini were deleted simultaneously. These results indicated that while the termini of the RSV N protein had a role in N-P binding they did not represent the binding site for P protein; elements from elsewhere on the N protein were involved in the binding of P protein. The termini may have a structural role; removal of one causes an unfavourable conformational change within the N protein preventing the N-P interaction. A second deletion at the other terminus results in a compensatory conformational rearrangement of the N protein allowing access to the P protein binding site on the RSV N protein. Our antibodies map to within, as is the case for αN009, or near, as with αN003, the domains deleted by Krishnamurthy and Samal. Blocking of the N-P interaction can be observed when N^{His} protein interacts with the monoclonal antibodies (see FIG. 1c). Capturing N^{His} protein using αN003 or αN009 prevented N^{His} from interacting with P^{His} protein. Similarly capture of N^{His} by immobilized p's protein appears to prevent access by the monoclonal antibodies yet the polyclonal antiserum (NRP14) to N protein shows that N^{His} has indeed been captured by the P^{His} protein (FIG. 1b). The RIPA in FIG. 2 confirms the blocking activity of the monoclonal antibodies. The N and P proteins are co-precipitated with the polyclonal antisera targeted against either the N protein (NRP14) or the P protein (PRP658). The monoclonal antibodies, however, do not co-immunoprecipitate P protein with N protein. A monoclonal antibody against the N protein, provided by G. Taylor (Institute of Animal Health, Compton, UK), also precipitates P with N protein (data not shown). Where this monoclonal antibody binds has not been determined. The proteins precipitated by αN003 and αN009 in FIG. 2 may represent RSV N protein that has not been complexed with P protein, or that the P protein has been displaced by the monoclonal antibodies. Displacement of P protein from N protein has previously been described for anti-hPIV1 N monoclonal antibodies (Ryan, Portner & Murti, 1993, Virology 193:376-384) The ELISA data in FIGS. 1b and 1c would not support a displacement explanation for the reciprocal blocking activity observed as we do not see exchange of the competing species. That the monoclonal antibodies epitopes also represent the P protein binding site can also be discounted. FIG. 1d represents an example of the data obtained when P^{His} and the monoclonals were used to bind N^{His} that had been immobilized on a microtitre plate. Irrespective of the order of addition of the competing ligands no blocking activity was observed suggesting independent binding sites probably on different molecules of immobilized N^{His}.

One interpretation is that the amino and carboxy termini of the N protein are folded near the P protein binding site. The reciprocal blocking that is observed when the monoclonal antibodies or P^{His} protein captures N^{His} protein is due to the close proximity of the monoclonal antibody binding domains and the P protein binding domain. Thus access is blocked for the competing ligand by steric hindrance (FIG. 1b and FIG. 1c). We cannot discount that the effect of binding one ligand could cause a conformational change within the N protein resulting in the masking of the binding site for another ligand. This data, with that of Krishnamurthy and Samal, would suggest that changes in the amino and carboxy termini of the RSV N protein would have an adverse effect on N-P binding possibly due to a conformational shift in the N protein preventing access to the P protein binding site.

A peptide scanning protocol was employed to identify sequences on the N protein involved in the RSV N-P binding interaction. The peptide binding data shows that P^{His} strongly binds to a subset of the N peptides, N4, N8, N11 and N17 (FIG. 3). The peptides span a region of approximately 314 amino acids of the N protein amino acid sequence (from a/a 46-360, FIG. 7) suggesting that the P protein binding site could be formed by the folding together of widely spaced regions of the N protein. The peptides that interacted with the P is protein were used in an attempt to determine if they could inhibit the N-P interaction in peptide blocking experiments. Unfortunately N8, N11 and N17 were unsuitable for blocking studies because of solubility problems that required the use of an acidic buffer (0.01% v/v acetic acid). Synthesis of shorter peptides covering the regions represented by N8, N11 and N17 may resolve the problems encountered. However, peptide N4 when used in a blocking assay prevented binding of N^{His} protein to immobilized P^{His} (FIG. 4). Peptide N4 may contain sequences responsible for binding P protein. Also this data confirms the observation from FIG. 1d that the monoclonal antibodies, although they block the N-P interaction do not bind to the same site as P protein.

Analysis of N-P interactions in other virus systems implicate a role for the N protein carboxy terminus in the N-P binding of Sendai virus, hPIVI and VSV.

We did not observe any significant binding of P protein to peptides in the carboxy terminus part of the N protein (FIG. 3). This would be in agreement with Krishnamurthy and Samal and Garcia-Barreno et al (J. Virol. 70:801-808, 1996), as a deletion of up to 39 amino acids of the carboxy terminus of the RSV N protein still retained P protein binding activity. Takacs et al (Proc. Natl. Acad. Sci. USA 90:10375-9, 1993) used the mammalian two-hybrid system to look at the VSV N protein interaction with P protein. The VSV N-P interaction was severely disrupted by deletion and/or mutagenesis of the final 10 amino acids of the VSV N protein carboxy terminus. However, binding of the VSV P protein to these amino acids was not demonstrated. Previous studies on Sendai virus, hPIV1 and the RSV N-P interaction relied on analysis where P protein binding is disrupted. Positive binding to these domains has not been proven experimentally. Deletion analysis may therefore be inducing conformational misfolding of the N proteins in these systems resulting in non-functional protein products. However, a role for the carboxy terminus of the N protein in N-P interaction is apparent. It may be involved in the direct binding of P protein (as implicated by the previous studies) but most certainly it has a structural role in maintaining the integrity of the P protein binding site on N protein. Insights of how the N protein interacts with both viral and cellular components may help in the understanding of the replication of the virus.

1. A compound which binds to respiratory syncytial virus (RSV) phosphoprotein, and which inhibits binding of said phosphoprotein to respiratory syncytial virus nucleocapsid protein.

2. A compound according to claim 1, wherein the compound binds to the carboxy terminus of RSV phosphoprotein.

3. A compound according to claim 1, wherein the compound binds to the C-terminal 20 amino acids of the phosphoprotein.

4. A compound according to claim 1, wherein the compound binds to a peptide having the sequence SEQ ID No. 1.

5. A compound according to claim 1, wherein the compound binds to a peptide having at least 75% sequence homology to SEQ ID No. 1.

6. A compound according to claim 5, wherein the compound binds to a peptide having at least 90% sequence homology to SEQ ID No. 1.

7. A compound according to claim 6, wherein the compound binds to a peptide having at least 95% sequence homology to SEQ ID No. 1.

8. A compound according to claim 1, wherein the compound comprises a peptide.

9. A compound according to claim 8, wherein the peptide comprises the amino acid sequence of SEQ ID No. 2.

10. A compound according to claim 8, wherein the peptide comprises a sequence having 80% homology to SEQ ID No. 2.

11. A compound according to claim 10, wherein the peptide comprises a sequence having 90% homology to SEQ ID No. 2.

12. A compound according to claim 11, wherein the peptide comprises a sequence having 95% homology to SEQ ID No. 2.

13. A compound according to claim 8, wherein the peptide comprises the amino acid sequence of SEQ ID No. 3.

14. A compound according to claim 8, wherein the peptide is selected from the group comprising the amino acid sequences of SEQ ID No. 4, 5, or 6, or sequences with conservative amino acid substitutions thereof.

15. A compound according to claim 1, wherein the compound comprises an antibody.

16. A method for treating respiratory syncytial virus infection comprising administering to a patient in suitable quantity a compound which binds to respiratory syncytial virus phosphoprotein, and which inhibits binding of said phosphoprotein to respiratory syncytial virus nucleocapsid protein.

17. A combination comprising a pharmacologically active amount of a compound which binds to respiratory syncytial virus phosphoprotein, and which inhibits binding of said phosphoprotein to respiratory syncytial virus nucleocapsid protein, in combination with a physiologically acceptable carrier.

18. A combination according to claim 17, wherein the active compound is enclosed in a lipid membrane.

19. A drug delivery device including a pharmacologically active amount of a compound which binds to respiratory syncytial virus phosphoprotein, and which inhibits binding of said phosphoprotein to respiratory syncytial virus nucleocapsid protein, enclosed in a lipid membrane, in combination with a physiologically acceptable carrier therefor.

20. A drug delivery device according to claim 19, wherein the delivery device is designed to administer a pharmacologically active amount of the compound to a patient's respiratory system.

21. A drug delivery device according to claim 20, comprising a metered inhaler device containing an aerosol formulation of said compound.

22. A kit for the detection of respiratory syncytial virus in a test sample, the kit comprising a compound which binds to respiratory syncytial virus phosphoprotein, and which inhibits binding of said phosphoprotein to respiratory syncytial virus nucleocapsid protein, in combination with reagents for detection of binding of the compound to the phosphoprotein.

23. A compound which binds to respiratory syncytial virus phosphoprotein, and which inhibits binding of said phosphoprotein to respiratory syncytial virus nucleocapsid protein, said compound being immobilised on a solid support substrate.

24. A recombinant cell expressing a peptide which binds to respiratory syncytial virus phosphoprotein, and which inhibits binding of said phosphoprotein to respiratory syncytial virus nucleocapsid protein.

25. Use of a peptide comprising the amino acid sequence of SEQ ID No. 1 in the identification of a compound which binds to respiratory syncytial virus phosphoprotein and which inhibits binding of said phosphoprotein to respiratory syncytial virus nucleocapsid protein.

26. Use of a peptide comprising an amino acid sequence having at least 75% sequence homology to that of SEQ ID No. 1 in the identification of a compound which binds to phosphoprotein of a virus related to respiratory syncytial virus.

27. Use of a peptide according to claim 26, wherein the amino acid sequence has at least 90% sequence homology to that of SEQ ID No. 1.

28. Use of a peptide according to claim 27, wherein the amino acid sequence has at least 95% sequence homology to that of SEQ ID No. 1.
